(19) United States
(12) Patent Application Publication
(10) Pub. No.: US 2026/0069515 A1
(43) Pub. Date: Mar. 12, 2026

(54) DISPERSION OF INORGANIC POWDER AND COMPOSITION FOR SUN PROTECTION COMPRISING THE SAME

(71) Applicant: AMOREPACIFIC CORPORATION, Seoul (KR)

(72) Inventors: Najeeb CHOOLAKADAVIL KHALID, Yongin-si (KR); Byoung Young WOO, Yongin-si (KR); Bongsoo PI, Yongin-si (KR); Youngkyu HAN, Yongin-si (KR); Heungsoo BAEK, Yongin-si (KR); Sungyeon CHO, Yongin-si (KR)

(73) Assignee: AMOREPACIFIC CORPORATION, Seoul (KR)

(21) Appl. No.: 19/310,377

(22) Filed: Aug. 26, 2025

(30) Foreign Application Priority Data
Sep. 12, 2024 (KR) 10-2024-0124713

(51) Int. Cl. A61K 8/27; A61K 8/02; A61K 8/06; A61K 8/34; A61K 8/36; A61K 8/58; A61K 8/73; A61Q 17/04

(52) U.S. Cl. CPC A61K 8/27; A61K 8/0241; A61K 8/062; A61K 8/345; A61K 8/361; A61K 8/585; A61K 8/731; A61K 8/735; A61Q 17/04; A61K 2800/33; A61K 2800/612; A61K 2800/623; A61K 2800/624; A61K 2800/63; A61K 2800/651

(57) ABSTRACT

The present disclosure relates to a dispersion of inorganic powder, and more specifically, the dispersion of inorganic powder includes a compound including a vicinal diol, a natural polymer, and an inorganic powder, which may exhibit excellent water dispersibility even without a surfactant, may be implemented in various formulations, may be easily applied to the skin, may minimize white cast phenomenon when applied to the skin, and may be easily washed with water after use.

### CROSS REFERENCE TO RELATED APPLICATION

The present application claims priority to Korean Patent Application No. 10-2024-0124713, filed Sep. 12, 2024, the entire contents of which are hereby incorporated by this reference.

### BACKGROUND OF THE INVENTION

### Field of the Invention

The present disclosure relates to a dispersion of inorganic powder and a composition for blocking ultraviolet (UV) including the same.

### Description of the Related Art

An inorganic ultraviolet (UV) blocker used in a cosmetic for blocking ultraviolet (UV) may exhibit phototoxicity due to dissociation. Therefore, conventionally, the inorganic ultraviolet (UV) blocker is surface-modified with a hydrophobic organic molecule (e.g., alkyl silane, stearic acid, etc.) to reduce the phototoxicity.

However, the inorganic ultraviolet (UV) blocker coated with the hydrophobic organic molecule may cause a white cast phenomenon when applied to the skin. In addition, the ultraviolet (UV) blocker coated with the hydrophobic organic molecule is not well dispersed in water, so there is a limitation in realizing various formulations, and it is conventionally manufactured in a water-in-oil (W/O) formulation, and after being applied to the skin, it is difficult to remove and has poor washability. To overcome these limitations, conventionally, a surfactant has been used to water-disperse the inorganic ultraviolet (UV) blocker.

### SUMMARY OF THE INVENTION

One aspect of the present disclosure is directed to providing a dispersion of inorganic powder.

One aspect of the present disclosure is directed to providing a composition for blocking ultraviolet (UV) including the dispersion of inorganic powder.

The dispersion of inorganic powder according to one aspect of the present disclosure includes a compound including a vicinal diol, a natural polymer, and inorganic powder. The composition for blocking ultraviolet (UV) according to one aspect of the present disclosure includes the dispersion of inorganic powder.

The dispersion of inorganic powder and/or the composition for blocking ultraviolet (UV) including the same according to one aspect of the present disclosure may be implemented in various formulations due to excellent dispersibility in aqueous phase.

In addition, the dispersion of inorganic powder and/or the composition for blocking ultraviolet (UV) including the same according to one aspect of the present disclosure may be easily applied to the skin, may minimize white cast phenomenon when applied to the skin, and may be easily washed with water after use. This is due to the excellent water dispersibility of the dispersion of inorganic powder and/or the composition for blocking ultraviolet (UV) including the same according to one aspect of the present disclosure.

In addition, the dispersion of inorganic powder and/or the composition for blocking ultraviolet (UV) including the same according to one aspect of the present disclosure may exhibit excellent water dispersibility even without a surfactant.

### BRIEF DESCRIPTION OF THE DRAWINGS

FIG. 1 illustrates results of comparing dispersibility of inorganic powder (ZnO) in various types of polyols.

FIG. 2 illustrates results of comparing water dispersibility of dispersion of inorganic powder of Comparative Example 1 (hydrophobic ZnO; left), Example 2 (hydrophobic ZnO+2,3 BG+CNF; center), and Example 6 (hydrophobic ZnO+2,3 BG+HA; right).

FIG. 3 illustrates results of confirming whether a water-soluble red dye is adsorbed to the dispersion of inorganic powder of Comparative Example 1 (hydrophobic ZnO; left), Example 2 (hydrophobic ZnO+2,3 BG+CNF; center), and Example 6 (hydrophobic ZnO+2,3 BG+HA; right). The water-soluble red dye appears as a portion of a dark black color in the upper and lower dishes of FIG. 3, and the state in which the water-soluble red dye is adsorbed to the dispersion of inorganic powder appears as a light gray color in the lower dishes of Examples 2 and 6 of FIG. 3.

FIG. 4 illustrates results of confirming cleansing power of a cosmetic composition including Comparative Example 1 (hydrophobic ZnO) (Comparative Example 6) and a cosmetic composition including Example 2 (hydrophobic ZnO+2,3 BG+CNF) (Example 9).

FIG. 5 illustrates results of confirming water dispersibility of the dispersion of inorganic powder of Comparative Example 7 (hydrophobic ZnO+water+CNF; left) and Comparative Example 8 (hydrophobic ZnO+water+HA; right).

### DETAILED DESCRIPTION OF THE INVENTION

Various embodiments of this document and the terms used in the embodiments are not intended to limit the technical features disclosed in this document to the particular embodiments and should be understood as including various alterations, equivalents, or alternatives of the corresponding embodiments.

The present inventors confirmed dispersibility of hydrophobic inorganic powder with respect to polyols having various structures (polyols having different positions of hydroxyl group (—OH)), and evaluated wetting characteristics, and confirmed that wetting characteristics of the hydrophobic inorganic powder are specifically improved only by a compound including a vicinal diol. Thereafter, by mixing the hydrophobic inorganic powder with a compound including a vicinal diol and a hydrophilic natural polymer, a ultraviolet (UV) blocker (dispersion of inorganic powder) that exhibits excellent water dispersibility even without a surfactant, is applicable to various formulations (e.g., O/W formulation, water-dispersible formulation, etc.), and is easily washable with water was developed.

### Definition of Terms

In the present disclosure, the term “dispersion of inorganic powder” refers to a material in which inorganic powder is dispersed, and its form is not limited. For example, the dispersion of inorganic powder may be a dispersion in which the inorganic powder is dispersed and/or wetted in a polyol (e.g., a compound including a vicinal diol). For example, the dispersion of inorganic powder may be a dispersion in which the inorganic powder is dispersed in a polyol (e.g., a compound including a vicinal diol) and a natural polymer. For example, the dispersion of inorganic powder may be a material in which the inorganic powder is dispersed and/or wetted in a polyol (e.g., a compound including a vicinal diol) and coated with a natural polymer. The dispersion may be a liquid or solid, but is not limited thereto.

In the present disclosure, the term “wetted inorganic powder” refers to a state in which the inorganic powder is wetted by a liquid or a state in which the inorganic powder is well dispersed in any material (e.g., a polyol), and its form is not limited. For example, the inorganic powder wetted with any first component (A) may be a state in which the inorganic powder is mixed with the first component (A) and becomes wetted. For example, the inorganic powder wetted with any first component (A) may be a state in which the inorganic powder is dispersed in the first component (A) and becomes wetted. In the present disclosure, the wetted inorganic powder may be a state in which the inorganic powder is mixed with a compound including a vicinal diol and becomes wetted. In the present disclosure, the wetted inorganic powder may be a form in which the inorganic powder is dispersed in a compound including a vicinal diol.

In the present disclosure, the term “compound including a vicinal diol” refers to a compound in which hydroxyl groups (—OH) are bonded to two adjacent atoms (e.g., carbon atoms). The two hydroxyl groups included in the compound including a vicinal diol occupy vicinal positions. That is, the two hydroxyl groups are bonded to adjacent atoms (e.g., carbon atoms).

For example, the compound including a vicinal diol may further include an additional hydroxyl group in addition to the two hydroxyl groups bonded to adjacent atoms (e.g., carbon atoms). In this case, the compound including a vicinal diol may be a polyol. For example, the compound including a vicinal diol may not further include an additional hydroxyl group other than the two hydroxyl groups bonded to adjacent atoms (e.g., carbon atoms). In this case, the compound including a vicinal diol may be a diol.

In the present disclosure, the term “silane” refers to a compound including substituted or unsubstituted SiH₄. The term “alkyl silane” refers to a silane substituted with at least one alkyl group. The alkyl silane may be substituted with additional substituents (e.g., alkoxy group) other than the alkyl group.

### Dispersion of Inorganic Powder

One aspect of the present disclosure provides a dispersion of inorganic powder.

In one embodiment, the dispersion of inorganic powder may include a compound including a vicinal diol, a natural polymer, and inorganic powder.

In one embodiment, the inorganic powder may be dispersed in the compound including a vicinal diol and the natural polymer. In one embodiment, the inorganic powder may be dispersed and wetted in the compound including a vicinal diol. In one embodiment, the natural polymer may be coating the inorganic powder. In one embodiment, the natural polymer may be coating the wetted inorganic powder. In one embodiment, in the dispersion of inorganic powder, the inorganic powder may be dispersed and wetted in the compound including a vicinal diol, and the wetted inorganic powder may be coated with the natural polymer.

In one embodiment, the dispersion of inorganic powder includes the inorganic powder, which is dispersed and wetted in the compound including a vicinal diol, and a natural polymer.

In one embodiment, the wetted inorganic powder may be coated with the natural polymer.

One aspect of the present disclosure provides a dispersion of inorganic powder, which includes the inorganic powder dispersed and wetted in the compound including a vicinal diol, and a natural polymer, in which the wetted inorganic powder is coated with the natural polymer.

The dispersion of inorganic powder according to one aspect of the present disclosure has excellent water dispersibility, and may be implemented in various formulations including an oil-in-water (O/W) formulation. In addition, due to the excellent water dispersibility, the dispersion of inorganic powder may be easily applied to the skin, and may be easily washed with water in the cleansing process after application.

In one embodiment, the compound including a vicinal diol may be at least one selected from the group consisting of 1,2-butylene glycol, 2,3-butylene glycol, 1,2-pentane diol, and 1,2-hexane diol, but is not limited thereto.

When the inorganic powder is dispersed in the compound including a vicinal diol (e.g., 1,2-butylene glycol, 2,3-butylene glycol, etc.) or dispersed and wetted by the compound including a vicinal diol, the coating efficiency of the surface of the wetted inorganic powder by the natural polymer may be excellent. On the other hand, when the inorganic powder is dispersed in a compound other than the compound including a vicinal diol (e.g., a polyol including two hydroxyl groups that are not vicinal, 1,3-butylene glycol) or dispersed and wetted by such other compound, the coating efficiency of the surface of the wetted inorganic powder by the natural polymer may not be high, and as a result, the water dispersibility of the dispersion of inorganic powder, which is a final product, may be poor.

In one embodiment, the inorganic powder may be at least one selected from the group consisting of zinc oxide (ZnO), titanium dioxide, and iron oxide, but is not limited thereto. In one embodiment, the inorganic powder may be a material having excellent ultraviolet (UV) blocking effect and proven safety.

In one embodiment, the inorganic powder is hydrophobic inorganic powder. The hydrophobic inorganic powder may have excellent water dispersibility by being wetted with a compound including a vicinal diol and coated with a natural polymer.

In one embodiment, the inorganic powder may be surface-modified with at least one hydrophobic compound selected from the group consisting of a silicone-based compound and a saturated fatty acid of C8 to C28.

In one embodiment, the silicone-based compound may be an alkyl silane, octyltriethoxysilane, or triethoxycaprylylsilane, but is not limited thereto. In one embodiment, the silicone-based compound may be octyltriethoxysilane or triethoxycaprylylsilane.

In one embodiment, the saturated fatty acid of C8 to C28 may be stearic acid, but is not limited thereto.

In one embodiment, the natural polymer is a hydrophilic natural polymer.

In one embodiment, the natural polymer may be at least one selected from the group consisting of cellulose, cellulose nanofibril (CNF), and hyaluronic acid, but is not limited thereto.

In one embodiment, the coating may be implemented by the natural polymer bonding to the compound including a vicinal diol, which is a component of the wetted inorganic powder. In one embodiment, the coating may be implemented by any functional group (e.g., carboxyl group, hydroxyl group, etc.) included in the natural polymer bonding to the compound including a vicinal diol, which disperses and/or wets the inorganic powder. In this case, the bonding may be a covalent bond, ionic bond, or hydrogen bond, but is not limited thereto.

In one embodiment, in the dispersion of inorganic powder, the wetted inorganic powder may have a surface thereof coated with the natural polymer.

In one embodiment, the dispersion of inorganic powder may include the compound including a vicinal diol in an amount of 10 to 50 wt % based on the total weight, but is not limited thereto. For example, the dispersion of inorganic powder may include the compound including a vicinal diol, based on the total weight, in an amount of 10 wt % or more, 15 wt % or more, 20 wt % or more, 25 wt % or more, 26 wt % or more, 27 wt % or more, 28 wt % or more, 29 wt % or more, 29.5 wt % or more, 30 wt % or more, 31 wt % or more, 32 wt % or more, 33 wt % or more, 34 wt % or more, 35 wt % or more, 40 wt % or more, 45 wt % or more, or 50 wt % or more, or 10 wt % or less, 15 wt % or less, 20 wt % or less, 25 wt % or less, 26 wt % or less, 27 wt % or less, 28 wt % or less, 29 wt % or less, 29.5 wt % or less, 30 wt % or less, 31 wt % or less, 32 wt % or less, 33 wt % or less, 34 wt % or less, 35 wt % or less, 40 wt % or less, 45 wt % or less, or 50 wt % or less, or may fall within a range combination thereof (e.g., 29 to 30 wt %, or 20 to 40 wt %), but is not limited thereto.

In one embodiment, the dispersion of inorganic powder may include the natural polymer in an amount of 0.01 to 10 wt % based on the total weight, but is not limited thereto. For example, the dispersion of inorganic powder may include the natural polymer, based on the total weight, in an amount of 0.01 wt % or more, 0.05 wt % or more, 0.1 wt % or more, 0.2 wt % or more, 0.3 wt % or more, 0.4 wt % or more, 0.45 wt % or more, 0.5 wt % or more, 0.55 wt % or more, 0.6 wt % or more, 0.7 wt % or more, 0.8 wt % or more, 0.9 wt % or more, 1 wt % or more, 2 wt % or more, 3 wt % or more, 4 wt % or more, 5 wt % or more, 6 wt % or more, 7 wt % or more, 8 wt % or more, 9 wt % or more, or 10 wt % or more, or 0.01 wt % or less, 0.05 wt % or less, 0.1 wt % or less, 0.2 wt % or less, 0.3 wt % or less, 0.4 wt % or less, 0.45 wt % or less, 0.5 wt % or less, 0.55 wt % or less, 0.6 wt % or less, 0.7 wt % or less, 0.8 wt % or less, 0.9 wt % or less, 1 wt % or less, 2 wt % or less, 3 wt % or less, 4 wt % or less, 5 wt % or less, 6 wt % or less, 7 wt % or less, 8 wt % or less, 9 wt % or less, or 10 wt % or less, or may fall within a range combination thereof (e.g., 0.4 to 0.6 wt %, or 0.1 to 1 wt %), but is not limited thereto.

In one embodiment, in the dispersion of inorganic powder, the weight ratio of the inorganic powder to the compound including a vicinal diol may be 1:0.1 to 1, but is not limited thereto. For example, in the dispersion of inorganic powder, the weight ratio of the inorganic powder to the compound including a vicinal diol may be 1:0.1 to 1, 1:0.1 to 0.9, 1:0.1 to 0.8, 1:0.2 to 0.7, 1:0.3 to 0.6, 1:0.4 to 0.5, or 1:0.41 to 0.43, but is not limited thereto.

In one embodiment, in the dispersion of inorganic powder, the weight ratio of the inorganic powder to the natural polymer may be 1:0.0001 to 0.1, but is not limited thereto. For example, in the dispersion of inorganic powder, the weight ratio of the inorganic powder to the natural polymer may be 1:0.0001 to 0.1, 1:0.0005 to 0.05, 1:0.001 to 0.01, 1:0.005 to 0.009, or 1:0.006 to 0.008, but is not limited thereto.

In one embodiment, in the dispersion of inorganic powder, the weight ratio of the compound including a vicinal diol to the natural polymer may be 1:0.001 to 1, but is not limited thereto. For example, in the dispersion of inorganic powder, the weight ratio of the compound including a vicinal diol to the natural polymer may be 1:0.001 to 1, 1:0.001 to 0.5, 1:0.005 to 0.1, 1:0.01 to 0.05, or 1:0.015 to 0.017, but is not limited thereto.

According to one embodiment, by wetting the hydrophobic inorganic powder with the compound including a vicinal diol, it is possible to enhance the coating efficiency of the surface of the hydrophobic inorganic powder by the hydrophilic polymer. As a result, the final product obtained by coating the surface of the wetted hydrophobic inorganic powder with the hydrophilic polymer may have excellent water dispersibility, may be implemented in various formulations, and may be easily washed with water when cleansing is required.

According to one embodiment, the hydrophobic inorganic powder may be effectively dispersed in the compound including a vicinal diol and the hydrophilic natural polymer, and the finally obtained dispersion of inorganic powder may have excellent water dispersibility, may be implemented in various formulations, and may be easily washed with water when cleansing is required.

### Composition for Blocking Ultraviolet (UV)

One aspect of the present disclosure provides a composition for blocking ultraviolet (UV) including the above-described dispersion of inorganic powder.

As for the dispersion of inorganic powder, it is the same as described above, and thus a detailed description thereof will be omitted.

In one embodiment, the composition for blocking ultraviolet (UV) may be a cosmetic composition, food composition, oral composition, non-therapeutic composition, non-therapeutic oral composition, pharmaceutical composition, or external application composition. Specifically, the composition for blocking ultraviolet (UV) may be an external application composition or a cosmetic composition, but is not limited thereto.

In one embodiment, the composition for blocking ultraviolet (UV) may be an oil-in-water (O/W) emulsion formulation or a water-dispersible formulation, but is not limited thereto.

In one embodiment, the composition for blocking ultraviolet (UV) may not include a surfactant, but is not limited thereto.

The dispersion of inorganic powder according to one aspect of the present disclosure has excellent water dispersibility by coating hydrophobic inorganic powder with a natural polymer that is not a surfactant, and thus may be well dispersed in the composition for blocking ultraviolet (UV) even without a surfactant. Meanwhile, conventionally, a surfactant was used together in order to water-disperse a hydrophobic inorganic powder.

In one embodiment, the composition for blocking ultraviolet (UV) may include the dispersion of inorganic powder in an amount of 1 to 50 wt % based on the total weight, but is not limited thereto. For example, the composition for blocking ultraviolet (UV) may include the dispersion of inorganic powder, based on the total weight, in an amount of 1 wt % or more, 5 wt % or more, 10 wt % or more, 15 wt % or more, 20 wt % or more, 25 wt % or more, 30 wt % or more, 34 wt % or more, 35 wt % or more, 40 wt % or more, 45 wt % or more, or 50 wt % or more, or 1 wt % or less, 5 wt % or less, 10 wt % or less, 15 wt % or less, 20 wt % or less, 25 wt % or less, 30 wt % or less, 34 wt % or less, 35 wt % or less, 40 wt % or less, 45 wt % or less, or 50 wt % or less, or may fall within a range combination thereof (e.g., 30 to 40 wt %, 25 to 45 wt %), but is not limited thereto.

In one embodiment, the composition for blocking ultraviolet (UV) may be applied once or multiple times to a subject requiring ultraviolet (UV) blocking, but is not limited thereto.

In one embodiment, the composition for blocking ultraviolet (UV) may be co-administered to a subject requiring ultraviolet (UV) blocking in combination with any other component capable of exhibiting ultraviolet (UV) blocking effects, but is not limited thereto.

In one embodiment, the composition for blocking ultraviolet (UV) may further include any other component capable of exhibiting ultraviolet (UV) blocking effects, but is not limited thereto.

In one embodiment, the composition for blocking ultraviolet (UV) may further include components that may be included in cosmetics. For example, the composition for blocking ultraviolet (UV) may further include additives such as preservatives, thickeners, viscosity modifiers, stabilizers, pearlizing agents, metal ion sequestrants, cationic surfactants, pH adjusters, fragrances, and dyes, and these may be easily purchased and used commercially.

In one embodiment, the composition for blocking ultraviolet (UV) may be provided in the form of toner, lotion, cream, serum, essence, eye cream, body lotion, body cream, powder, ointment, spray, or concealer stick, but is not limited thereto.

### Method of Blocking Ultraviolet (UV)

One aspect of the present disclosure provides a method of blocking ultraviolet (UV) including: treating a dispersion of inorganic powder to a subject.

One aspect of the present disclosure provides a method of blocking ultraviolet (UV) including: treating a composition including a dispersion of inorganic powder to a subject.

One aspect of the present disclosure provides a method of protecting the skin of a subject from ultraviolet (UV) including: treating a dispersion of inorganic powder to a subject.

One aspect of the present disclosure provides a method of protecting the skin of a subject from ultraviolet (UV) including: treating a composition including a dispersion of inorganic powder to a subject.

As for the dispersion of inorganic powder, it is the same as described above, and thus a detailed description thereof will be omitted.

In one embodiment, the subject may be an animal including a mammal (e.g., a human), but is not limited thereto.

In one embodiment, the subject may be a subject requiring ultraviolet (UV) blocking. In one embodiment, the subject may be a subject requiring a dispersion of inorganic powder or a composition including the same.

In the present disclosure, the term “treating” may include administration or application to the skin. For example, the administration may be an administration through oral administration, transdermal administration, subcutaneous administration, intravenous administration, intraperitoneal administration, intramuscular administration, application, topical application, or the like, but is not limited thereto.

In one embodiment, a daily amount of treatment of a dispersion of inorganic powder or the composition including the dispersion of inorganic powder may be 0.0001 to 10000 mg/kg, but is not limited thereto. It should be understood that an amount of treatment of the dispersion of inorganic powder or the composition including the dispersion of inorganic powder needs to be determined in light of various related factors such as the route of administration, age, gender, and body weight of the subject, and therefore, the amount of treatment shall in no way limit the scope of the present invention.

In one embodiment, a dispersion of inorganic powder or the composition including the dispersion of inorganic powder may be treated to the subject once daily or in multiple divided doses per day, but is not limited thereto.

In one embodiment, a dispersion of inorganic powder or the composition including the dispersion of inorganic powder may be co-treated in combination with any other component capable of exhibiting a skin improvement effect and/or ultraviolet (UV) blocking effect. The specific type of any other component is not limited.

### Use of Dispersion of Inorganic Powder

One aspect of the present disclosure provides a use of the above-described dispersion of inorganic powder.

One aspect of the present disclosure provides a use of the above-described dispersion of inorganic powder for manufacturing a composition for blocking ultraviolet (UV).

One aspect of the present disclosure provides a use of the above-described dispersion of inorganic powder for use in ultraviolet (UV) blocking.

As for the dispersion of inorganic powder, it is the same as described above, and thus a detailed description thereof will be omitted.

Hereinafter, in order to describe the present invention more specifically, examples will be given in detail. The following examples are provided for illustrative purposes only to aid in understanding the present invention, and are not intended to limit the technical teachings and scope of the present invention.

### Experimental Example 1: Evaluation of Wetting of Hydrophobic Inorganic Powder

A dispersion test was conducted by mixing a hydrophobic ZnO powder coated with alkyl silane with each of six types of different polyols.

The hydrophobic ZnO powder coated with alkyl silane was prepared using ZnO coated with octyltriethoxysilane (OTS) (product name: MZX-508 OTS; manufacturer: Tayca). Specifically, 2 g of hydrophobic ZnO powder coated with alkyl silane (MZX-508 OTS) was added to 18 g of each of the six types of polyols described in Table 1 below and mixed for 3 minutes at 300 rpm using an Agitation-mixer.

TABLE 1

| | | Dispersion |
|---|---|---|
| | | result |
| | | (dispersibility |
| | | of inorganic |
| Classification | Polyol mixed with inorganic powder | powder) |
| Reference | 1,3-propyleneglycol (hereinafter, | Dispersed X |
| Example 1 | 1,3 PG) | |
| Reference | 1,3-butylene glycol (hereinafter, | Dispersed X |
| Example 2 | 1,3 BG) | |
| Reference | 1,2-butylene glycol (hereinafter, | Dispersed O |
| Example 3 | 1,2 BG) | |
| Reference | 2,3-butylene glycol (hereinafter, | Dispersed O |
| Example 4 | 2,3 BG) | |
| Reference | 1,2-pentane diol (hereinafter, | Dispersed O |
| Example 5 | 1,2 PD) | |
| Reference | 1,2-hexane diol (hereinafter, | Dispersed O |
| Example 6 | 1,2 HD) | |

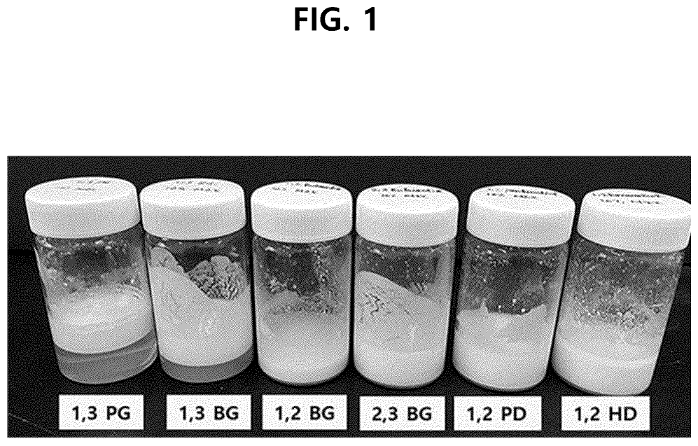

As illustrated in Table 1 and FIG. 1, in polyols such as 1,3 PG and 1,3 BG, which do not have vicinal diols, the hydrophobic ZnO powder was not dispersed. In contrast, in polyols having vicinal diols, the hydrophobic ZnO powder was effectively dispersed.

### Experimental Example 2: Manufacture of Dispersion of Inorganic Powder (Examples and Comparative Examples) and Evaluation of Water Dispersibility

Hydrophobic ZnO powder, coated with alkyl silane, was wetted in each of six types of different polyols, followed by coating with a hydrophilic natural polymer, to manufacture the dispersion of inorganic powder. Thereafter, the water dispersibility of the manufactured dispersion of inorganic powder was evaluated. The detailed method is described below.

### 1. Manufacture of Dispersion of Inorganic Powder (Examples and Comparative Examples)

ZnO coated with alkyl silane (product name: MZX-508 OTS; manufacturer: Tayca) was wetted in each of six types of different polyols (1,3 PG, 1,3 BG, 1,2 BG, 2,3 BG, 1,2 PD, and 1,2 HD) and coated with a hydrophilic natural polymer (cellulose nanofibril (hereinafter, CNF) or hyaluronic acid (hereinafter, HA)), to obtain the dispersion of inorganic powder (Examples 1 to 8 and Comparative Examples 2 to 5). Specifically, to proceed with wetting with polyol and coating with the natural polymer, the natural polymer was first dispersed in the polyol, and then the hydrophobic ZnO (ZnO coated with alkyl silane) inorganic powder was slowly added while performing agitation-mixing at 300 rpm. The wetting with polyol and the coating with the natural polymer are performed simultaneously. The natural polymer may be in an amount of 0.1 to 0.5 wt %, the hydrophobic ZnO (ZnO coated with alkyl silane) inorganic powder may be up to 70 wt %, and the polyol may be 29.5 to 49.9 wt %. The final product was in the form of a powder or a paste, and drying was not performed.

In addition, to confirm whether the coating with the natural polymer may be performed smoothly without wetting with polyol, instead of the polyol, CNF (Comparative Example 7) or HA (Comparative Example 8) was dispersed in water, followed by slow addition of hydrophobic ZnO (ZnO coated with alkyl silane) inorganic powder while performing agitation-mixing at 300 rpm.

Table 2 shows the components constituting the dispersion of inorganic powder in the examples and comparative examples. In Table 2, for Examples 1 to 8 and Comparative Examples 2 to 5, the content of the components constituting the dispersion of inorganic powder may be 70 wt % of hydrophobic ZnO (ZnO coated with alkyl silane), 29.5 wt % of polyol, and 0.5 wt % of natural polymer, based on the total weight. Comparative Example 1 is hydrophobic ZnO (product name: MZX-508 OTS) that is neither wetted with the polyol nor coated with the natural polymer. In Table 2, for Comparative Examples 7 and 8, the content of the components constituting the dispersion of inorganic powder may be 70 wt % of hydrophobic ZnO (ZnO coated with alkyl silane), 29.5 wt % of water, and 0.5 wt % of natural polymer, based on the total weight.

TABLE 2

| | | | | Water |
|---|---|---|---|---|
| | | | | Dispersibility |
| | Inorganic | | Natural | Evaluation |
| Classification | powder | Polyol | polymer | Result |
| Comparative | hydrophobic | X | X | Phase |
| Example 1 | ZnO | | | separation |
| Comparative | | 1,3 PG | CNF | Phase |
| Example 2 | | | | separation |
| Comparative | | 1,3 BG | | Phase |
| Example 3 | | | | separation |
| Example 1 | | 1,2 BG | | Dispersed |
| Example 2 | | 2,3 BG | | Dispersed |
| Example 3 | | 1,2 PD | | Dispersed |
| Example 4 | | 1,2 HD | | Dispersed |
| Comparative | | 1,3 PG | HA | Phase |
| Example 4 | | | | separation |
| Comparative | | 1,3 BG | | Phase |
| Example 5 | | | | separation |
| Example 5 | | 1,2 BG | | Dispersed |
| Example 6 | | 2,3 BG | | Dispersed |
| Example 7 | | 1,2 PD | | Dispersed |
| Example 8 | | 1,2 HD | | Dispersed |
| Comparative | Hydrophobic | X (replaced | CNF | Phase |
| Example 7 | ZnO | with water) | | separation |
| Comparative | Hydrophobic | X (replaced | HA | Phase |
| Example 8 | ZnO | with water) | | separation |

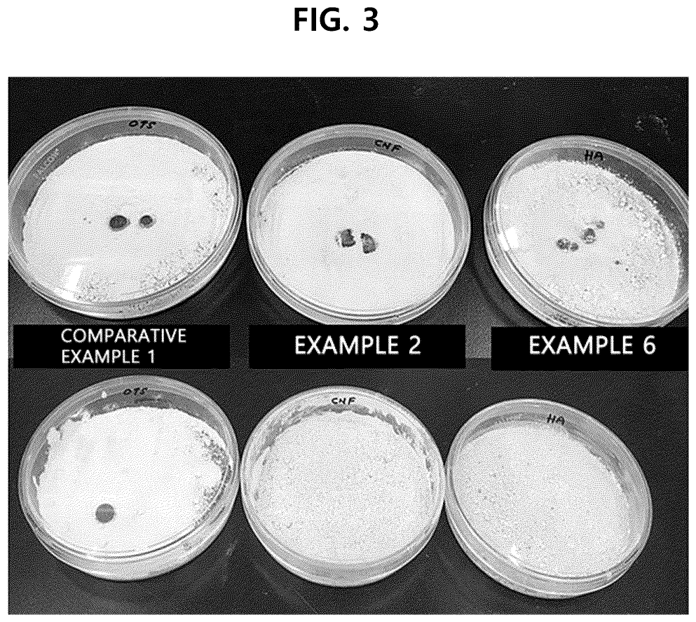

In the dispersion of inorganic powder, in order to confirm whether the surface of the hydrophobic ZnO inorganic powder was effectively coated with a hydrophilic material, a water-soluble red dye (TPP RED NO. 227, labeling name: RED 33 (CI 17200), 2,7-Naphthalenedisulfonic acid, 5-amino-4-hydroxy-3-(phenylazo), disodium salt) was added to each of the dispersions of inorganic powder manufactured by the above-described manufacturing method, followed by mixing, and it was confirmed whether the red dye was adsorbed on the powder. As illustrated in FIG. 3, the water-soluble red dye was adsorbed onto the surface of the dispersions of inorganic powder of Examples 1 to 8. In contrast, the water-soluble red dye was not adsorbed onto the surface of the hydrophobic ZnO powder of Comparative Example 1, which was not wetted with the polyol and not coated with the natural polymer (the leftmost in FIG. 3). In FIG. 3, the upper image is an image immediately after the addition of the water-soluble red dye (the black colored portions within the upper three dishes in FIG. 3 are the water-soluble red dye), and the lower image is an image after a predetermined period of time has elapsed following the addition of the water-soluble red dye (among the lower dishes of FIG. 3, the dish of Comparative Example 1 shows a state in which the water-soluble red dye has not adsorbed onto the surface of the powder (shown in black)). Among the lower dishes of FIG. 3, the dishes of Examples 2 and 6 show a state in which the water-soluble red dye (shown in black in FIG. 3) was well adsorbed onto the surface of the powder (appearing in light gray). In addition, Comparative Examples 7 and 8, in which the hydrophobic ZnO inorganic powder was obtained by being mixed with the polymer without the polyol, did not have the hydrophilic polymer effectively coated onto the hydrophobic ZnO inorganic powder.

2. Evaluation of Water Dispersibility of Dispersion of Inorganic Powder (Examples and Comparative Examples) The water dispersibility of the dispersions of inorganic powder obtained in “Experimental Example 2.1. Manufacture of dispersion of inorganic powder” was evaluated.

Specifically, using an Agitation-mixer (300 rpm, 10 minutes), the dispersions of inorganic powder of Examples 1 to 8, Comparative Examples 1 to 5, and Comparative Examples 7 and 8 were dispersed in water such that the concentration of hydrophobic ZnO became 10% (that is, the powder of each Example or Comparative Example was dispersed in 45 mL of water such that 5 g of hydrophobic ZnO was mixed).

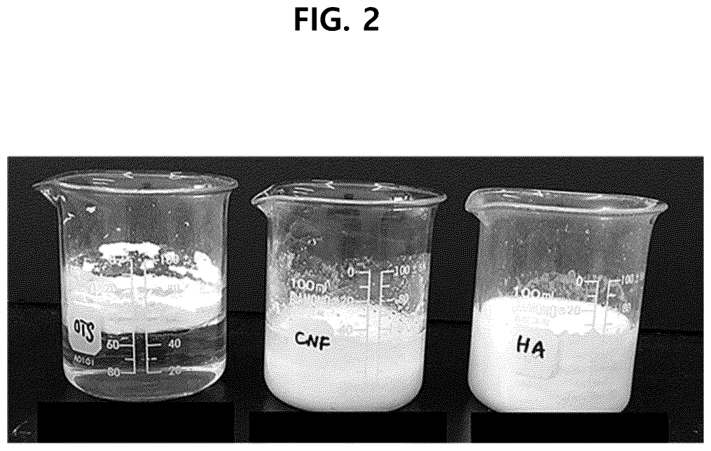

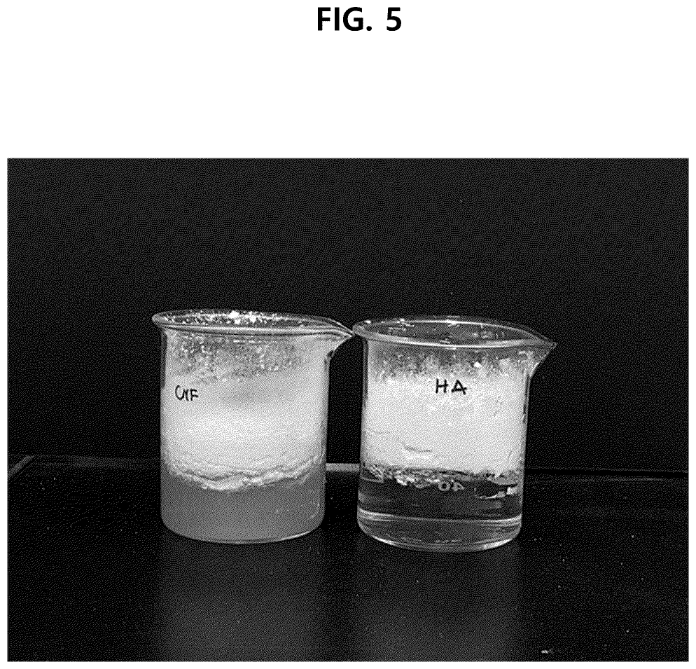

The results of water dispersibility evaluation for the Examples and Comparative Examples are disclosed in Table 2 (far right column). As shown in Table 2 and FIG. 2, the dispersions of inorganic powder (Examples 1 to 8) obtained by coating hydrophobic ZnO powder, wetted by being dispersed in the polyol having a vicinal diol, with a natural polymer (CNF or HA) exhibited excellent water dispersibility. In contrast, the dispersions of inorganic powder (Comparative Examples 2 to 5) obtained by coating hydrophobic ZnO powder, wetted using the polyol not having a vicinal diol such as 1,3 PG and 1,3 BG, with the natural polymer (CNF or HA) showed poor dispersibility in water and underwent phase separation. Additionally, as illustrated in FIG. 2, the hydrophobic ZnO powder (Comparative Example 1), which was neither wetted with the polyol nor coated with the natural polymer, did not disperse well in water (leftmost in FIG. 2). As illustrated in FIG. 5, the dispersions (Comparative Examples 7 and 8) obtained by mixing hydrophobic ZnO powder with a natural polymer without the polyol did not disperse well in water and underwent phase separation (see FIG. 5: left image corresponds to Comparative Example 7, and right image to Comparative Example 8).

Experimental Example 3: Manufacture of cosmetic composition for blocking ultraviolet (UV) including dispersion of inorganic powder and evaluation of cleansing performance improvement A cosmetic composition for blocking ultraviolet (UV), including hydrophobic ZnO, and a cosmetic composition for blocking ultraviolet (UV), including the dispersions of Examples in Table 2, were manufactured using the compositions listed in Table 3, and their cleansing power was compared. In Table 3, the hydrophobic ZnO corresponds to Comparative Example 1, and the hydrophilic ZnO corresponds to Example 2.

TABLE 3

| | | Example 9 |
|---|---|---|
| | Comparative Example 6 | (cosmetic |
| Composition (wt %) | (cosmetic composition) | composition) |
| Water (D.I. Water) | to 100 | to 100 |
| Disodium EDTA | 0.05 | 0.05 |
| 1,3-Butylene glycol | 5 | 5 |
| Xanthan Gum | 1 | 1 |
| Hydrophobic ZnO | 24 | 0 |
| (MZX508-OTS: Zinc oxide, | | |
| Triethoxycaprylylsilane) | | |
| Hydrophilic ZnO | 0 | 34.3 |
| [Hydrophobic ZnO | | |
| (MZX508-OTS) 70 wt %, 2,3 | | |
| BG 29.5 wt %, CNF 0.5 wt %] | | |
| Butyloctyl salicylate | 5 | 5 |
| Cetearyl Olivate, Sorbitan | 3 | 3 |
| Olivate | | |
| (Weight ratio of cetearyl | | |
| olivate to sorbitan olivate = | | |
| 60:40) | | |
| Cleansing power | 38.75% | 97.61% |

The cosmetic composition for blocking ultraviolet (UV) manufactured with the compositions in Table 3 was applied to the skin (an area of 3×3 cm in size was used as the test site) at 2 ug/cm² (that is, the cosmetic composition was applied at 18 ug to the test site of 9 cm², which is 3×3 cm in size), and then washed with water after a predetermined period of time to evaluate the cleansing power. After cleansing, the average skin tone brightness (L*) value at each point in time of the corresponding test site was analyzed, and the cleansing rate (%) was calculated using Equation 1 below.

Cleansing rate (%)=(Skin tone brightness (L*) value after use of cosmetic composition−Skin tone brightness (L*) value after cleansing with cleansing foam)/(Skin tone brightness (L*) value after use of cosmetic composition−Skin tone brightness (L*) value before use of cosmetic composition)×100 [Equation 1]

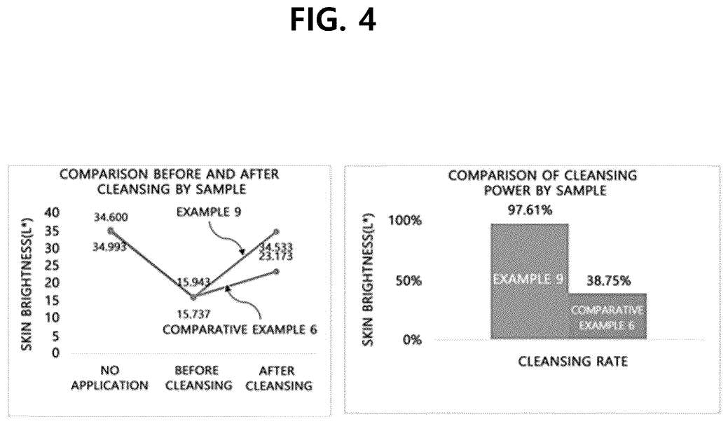

As a result, the cleansing power of the cosmetic composition of Comparative Example 6 including hydrophobic ZnO was poor, at 38.75%. In contrast, the cleansing power of the cosmetic composition of Example 9, including the dispersion of inorganic powder (that is, a form in which hydrophobic ZnO inorganic powder is dispersed in a compound including a vicinal diol and a natural polymer; hydrophilic ZnO), was excellent, at 97.61% (FIG. 4).

What is claimed is:

1. A dispersion of inorganic powder, comprising: a compound including a vicinal diol, a natural polymer, and an inorganic powder.

2. The dispersion of inorganic powder of claim 1, wherein, in the dispersion of inorganic powder, the inorganic powder is dispersed and wetted in the compound including the vicinal diol, and the wetted inorganic powder is coated with the natural polymer.

3. The dispersion of inorganic powder of claim 1, wherein the compound including the vicinal diol is at least one selected from the group consisting of 1,2-butylene glycol, 2,3-butylene glycol, 1,2-pentane diol, and 1,2-hexane diol.

4. The dispersion of inorganic powder of claim 1, wherein the inorganic powder is at least one selected from the group consisting of zinc oxide, titanium dioxide, and iron oxide.

5. The dispersion of inorganic powder of claim 1, wherein the inorganic powder is a hydrophobic inorganic powder.

6. The dispersion of inorganic powder of claim 1, wherein the inorganic powder is surface-modified with at least one hydrophobic compound selected from the group consisting of a silicone-based compound and a saturated fatty acid of C8 to C28.

7. The dispersion of inorganic powder of claim 6, wherein the silicone-based compound is alkyl silane, octyltriethoxysilane, or triethoxycaprylylsilane.

8. The dispersion of inorganic powder of claim 6, wherein the saturated fatty acid of C8 to C28 is stearic acid.

9. The dispersion of inorganic powder of claim 1, wherein the natural polymer is a hydrophilic natural polymer.

10. The dispersion of inorganic powder of claim 1, wherein the natural polymer is at least one selected from the group consisting of cellulose, cellulose nanofibril, and hyaluronic acid.

11. The dispersion of inorganic powder of claim 1, wherein the dispersion of inorganic powder comprises the compound including the vicinal diol in an amount of 10 to 50 wt %, based on a total weight.

12. The dispersion of inorganic powder of claim 1, wherein the dispersion of inorganic powder comprises the natural polymer in an amount of 0.01 to 10 wt % based on a total weight.

13. A method of protecting skin of a subject from ultraviolet (UV), comprising:
treating a subject requiring ultraviolet (UV) blocking with a composition comprising the dispersion of inorganic powder of claim 1.

14. The method of claim 13, wherein the composition is an external application composition or a cosmetic composition.

15. The method of claim 13, wherein the composition is an oil-in-water (O/W) emulsion formulation or a water-dispersible formulation.

16. The method of claim 13, wherein the composition does not comprise a surfactant.

17. The method of claim 13, wherein the composition comprises the dispersion of inorganic powder in an amount of 1 to 50 wt %, based on a total weight.
